(19) United States
(12) Patent Application Publication
(10) Pub. No.: US 2018/0296202 A1
(43) Pub. Date: Oct. 18, 2018

(54) Contra Retractor

(71) Applicant: Jeffrey Boutte, Wylie, TX (US)

(72) Inventor: Jeffrey Boutte, Wylie, TX (US)

(21) Appl. No.: 15/488,012

(22) Filed: Apr. 14, 2017

(51) Int. Cl. A61B 17/02; A61B 1/07; A61B 1/32

(52) U.S. Cl. CPC A61B 17/02; A61B 2017/00473; A61B 1/32; A61B 1/07

(57) ABSTRACT

A handheld surgical retractor device for manually effecting safe and certain contralateral displacement of a patient's incised tissue by a single surgical assistant during invasive surgery, while said assistant manually effects lateral displacement of the patient's incised tissue to create an operative field. The device includes a handle attached to a first bar for transmitting a compressive force to a second bar to generate a tensile force acting on a retractor blade, with minimum obscuration and obstruction of the operative field. A light pipe may be attached for use in illuminating the operative field. Another device embodiment allows selection of removable retractor blades to match the characteristics of the blade based on the surgical requirements.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to tissue retractor devices for invasive surgery on a patient.

### Description of Related Art Including Information Disclosed Under 37 CFR 1.97 and 1.98

In performing invasive surgical procedures on a patient, use of a retractor is often desirable or necessary to enlarge an operative field sufficiently for surgical access. Modern tissue retractor devices, such as that depicted in FIG. 1, provide a retractor blade (102) attached to a simple handle (104) for gripping by a surgical assistant. The retractor blade (102) is placed within the incision where it engages tissue on the edge of the incision, whereupon the assistant grips the handle (104) and pulls, thereby imparting a tensile force that is transferred to the retractor blade, causing the tissue edge to retract and the incision to open laterally. However, to fully open the operative field for access by the surgeon it is often necessary to utilize a second retractor device on the patient side opposite that of the surgical assistant.

To utilize a second retractor device, it is usually necessary to have a second surgical assistant standing on the side of the patient opposite that of the first surgical assistant. The second surgical assistant, likewise, grips the second retractor handle and pulls, imparting a tensile force on the handle that subsequently causes the blade engaging the tissue edge to retract the tissue, thereby causing the incision to open laterally. While this creates a wider operative field for the surgeon, the surgeon's ability to move about on either side of the patient is now adversely affected by the presence of the second surgical assistant.

The participation of a second surgical assistant increases overall surgery costs dramatically. Also, scheduling of surgical procedures can become difficult when the number of participants increases. To reduce the need for a second surgical assistant it is sometimes possible for the first surgical assistant to reach across the patient to operate the second retractor device. However, this is highly impractical especially with large patients that can be difficult to span, which can also result in the surgical assistant obstructing the operative field. Operating a standard retractor in this fashion by reaching across the patient is also fatiguing for the assistant especially during lengthy surgical procedures, and can cause excessive physical stress and discomfort that limits the effectiveness of the assistant. What is needed is a retractor device that allows a single surgical assistant to retract incised tissue on the side of a patient opposite that of where the assistant is stationed, with minimal obscuration and obstruction of the operative field.

### BRIEF SUMMARY OF THE INVENTION

Embodiments of the invention include a retractor apparatus comprising a handle member for gripping by an operator standing on a lateral side of a patient, the handle member connected by a plurality of beam members to a retractor blade member, the retractor blade member having a tissue engagement face for engaging an incised tissue on a contralateral side of the patient, the plurality of beam members configured such that the handle member is oriented on the lateral side of the patient and is operable on the blade member in the incised tissue, which is oriented on the contralateral side of the patient, the contralateral incised tissue displaced in a contralateral direction when the handle member is pushed by the operator. In another embodiment one or more of the plurality of beam members is adjustable in length. In another embodiment, the length of the shear beam member is adapted to achieve the desired surgical field opening. Additional embodiments of the apparatus comprise: a shear beam member connecting a compressive beam member to a tensile beam member, whereby a compressive force generated by the pushing motion is transferred as a shear force onto the shear beam member, and whereby the shear force is transferred as the tensile force onto the tensile beam member where the tensile force acts upon the blade member to impart force upon the contralateral incised tissue; a light pipe attached to the tensile beam member and adapted to accept a light emitted from a light source and to direct the emitted light downward along a back face of the retractor blade; a light pipe attached to the tensile beam member and adapted to accept a light emitted from a light source and to direct the emitted light downward along a back face of the retractor blade, the light pipe coupled to an optical fiber for transmitting emitted light from a light emission source; and/or a releasable retention member for releasably retaining the retractor blade on the tensile beam member.

An additional embodiment of the retractor apparatus comprises: at least one retractor blade member having and a back face and an opposing tissue engagement face, the tissue engagement face for engaging an incised tissue of a patient; and a handle member for gripping, the handle member connected to the at least one retractor blade member through a plurality of beam members, the plurality of beam members including a compressive beam member and a tensile beam member configured such that a user pushing on the handle member imparts a compressive force onto the compressive beam member, which is transferred as a tensile force onto the tensile beam member to act on the retractor blade member thereby imparting force on an engaged tissue of a patient. In another embodiment one or more of the plurality of beam members is adjustable in length. In yet another embodiment the apparatus comprises a shear beam member connecting the compressive beam member to the tensile beam member, whereby the compressive force is transferred as a shear force onto the shear beam member, and whereby the shear force is transferred as the tensile force onto the tensile beam member. Still other embodiments further comprise: a shear beam member connecting the compressive beam member to the tensile beam member, wherein the length of the shear beam member is adapted to achieve the desired surgical field opening; a light pipe attached to the tensile beam member and adapted to accept light emitted from a light source and to direct the emitted light downward along the back face of the retractor blade; a light pipe attached to the tensile beam member and adapted to accept light emitted from a light source and to direct the emitted light downward along the back face of the retractor blade, the light pipe coupled to an optical fiber for transmitting emitted light from a light emission source; and/or a releasable retention member for releasably retaining the retractor blade on the tensile beam member.

An additional embodiment of the retractor apparatus comprises: a handle member for gripping, the handle member connected to a first beam member, the first beam member connected to a second beam member, and the second beam member connected to a third beam member, the third beam member attached to at least one retractor blade member, the retractor blade member having a back face an opposing tissue engagement face for engaging the tissue of an incision and, wherein the first, second, and third beam members are configured such that when the retractor blade member tissue engagement face is engaged with an incised tissue on a patient, a compressive force imparted with the handle member onto the first beam member is transferred as a shear force onto the second beam member and is subsequently transferred as a tensile force onto the third beam member to act on the retractor blade member thereby imparting force on the engaged tissue. In another embodiment one or more of the beam members is adjustable in length. In yet another embodiment the length of the second beam member is adapted to achieve the desired surgical field opening. In yet another embodiment the apparatus further comprises: a light pipe attached to the third beam member and adapted to accept light emitted from a light source and to direct the emitted light downward along the back face of the retractor blade; a light pipe attached to the third beam member and adapted to accept light emitted from a light source and to direct the emitted light downward along the back face of the retractor blade, the light pipe coupled to an optical fiber for transmitting emitted light from a light emission source; and/or a releasable retention member for releasably retaining the retractor blade on the third beam member.

### BRIEF DESCRIPTION OF THE SEVERAL VIEWS OF THE DRAWING(S)

The present invention will be more fully understood by reference to the following detailed description of the preferred embodiments of the present invention when read in conjunction with the accompanying drawings, wherein:

FIG. 1 is a depiction of a typical bladed tissue retractor device;

FIG. 2 is a depiction of a first embodiment of the contra retractor device as disclosed and claimed herein;

FIG. 3 is a bottom perspective view of the embodiment showing the physical structure not visible in the previous figure;

FIG. 4 is a depiction of another embodiment of the invention, highlighting use of a light pipe and support structure for illuminating an operative field;

FIG. 5 is a depiction of another embodiment of the invention; highlighting an alternate handle and blade configuration; and

FIG. 6 is a depiction of use of the invention to establish an operative field.

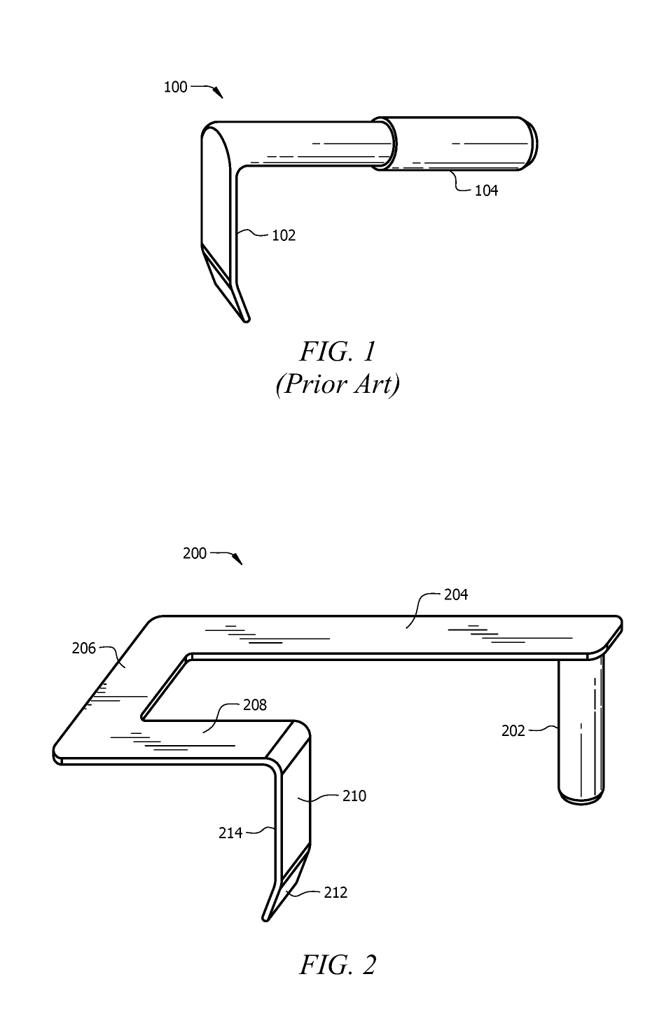

The above figures are provided for illustration and description only, and are not intended to define the limits of the disclosed invention. Use of the same reference number in multiple figures is intended to designate the same or similar parts. Furthermore, if, and when, the terms “top,” “bottom,” “first,” “second,” “upper,” “lower,” “height,” “width,” “length,” “end,” “side,” “horizontal,” “vertical,” and similar terms are used herein, these terms have reference only to the structure shown in the drawing and are utilized only to facilitate describing the specific embodiment. The extension of the figures with respect to number, position, relationship, and dimensions of the parts to form the preferred embodiment will be explained or will be within the skill of the art after the following teachings of the present invention have been read and understood.

### DETAILED DESCRIPTION OF THE INVENTION

FIG. 2 depicts a first embodiment of the contra retractor device as disclosed and claimed herein. The device (200) is intended for manual operation by a surgical assistant or other operator. A handle member (202) for gripping by the operator is attached to a first beam member (204). The first beam (204) is connected through a second beam member (206) to a third beam member (208), which is connected to a retractor blade member (210) as shown. The retractor blade tip (212) is bent slightly in the direction of intended force to resist slippage of the blade tissue engagement face (214) in relation to the retracted tissue, when force is applied to the device in operation.

The device (200) configuration is such that a compressive force applied to the first beam member (204) via the handle (202) is transferred to the second beam member (206) where it becomes a shear force, which is subsequently transferred to the third beam member (208) as a tensile force when the blade member (210) is held stationary or otherwise has its movement resisted by a physical object. The first beam member (204) is also referred to herein as the “compressive beam member.” The second beam member (206) is also referred to herein as the “shear beam member.” The third beam member (208) is also referred to herein as the “tensile beam member.”

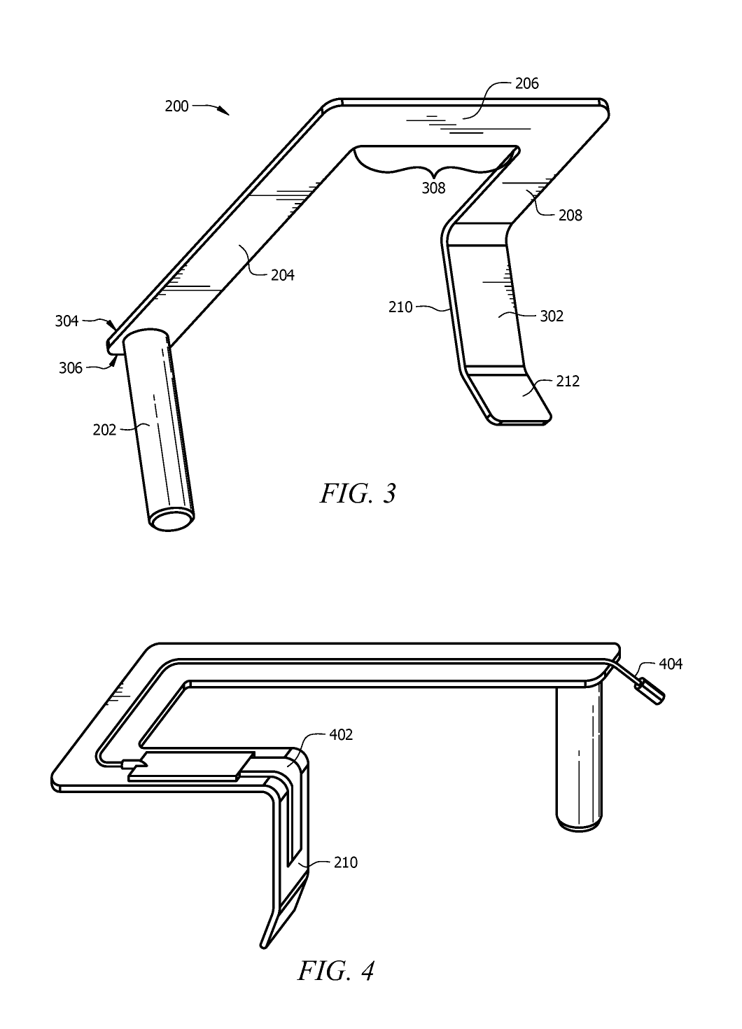

FIG. 3 presents a bottom perspective view of the embodiment showing the physical structure not visible in the previous figure. Visible is the underside surface of the first beam member (204), the second beam member (206), and third beam member (208), as well as the tissue engagement face (302) of the retractor blade (210). The tissue engagement face (302) is opposite that of the back face of the device.

The handle member (202) in this embodiment is permanently attached to the first beam member (204) by welding, but in other embodiments may be removable. In yet another embodiment the handle may also be placed on the top surface (304) or the forward edge (306) of the first beam member (204) to afford greater clearance of the device over the body of a patient on which it is being utilized. Moreover, the chosen length (308) of the second beam member (206) defines the space between the first and third beam members (204 and 208), which is beneficial in maintaining a clear view of, and access to, the surgical field. Thus, the length of the second beam member is adapted to the requirements of the surgical field opening. In another embodiment the second beam member, or shear beam member, (206) is segmented and thereby adjustable in length to adapt the device to the requirements of the surgical field opening.

The device (200) is intended to be rigid to resist deformation, and may be formed from a metal, composite, or other biocompatible material. Use of biocompatible metal is preferable given its corrosion resistance, low bioreactivity, and ability to be disinfected or sterilized. For example, cobalt-chrome, titanium, stainless steel, tantalum, and the like are useful. Use of composite materials may be suitable for construction of disposable contra retractor devices or portions of such devices. The device (200) may be formed from a single material, or may combine materials as desired. For example, metal may be utilized for the entire rigid structure with a polymer handle or device coating. Moreover, the device may be formed in a single or multiple sections based upon manufacturability constraints. One of ordinary skill will appreciate that such decisions are commonly made and implemented.

Lighting of the operative field is beneficial for the surgeon and assistant as it affords a better view of exposed tissue during surgery. FIG. 4 is a depiction of another embodiment of the invention, highlighting use of a light pipe and support structure for illuminating an operative field. As shown, a lighting member includes a light pipe (402), which is an optical fiber or solid transparent polymer light wave guide that transmits light from a light emission source to a desired location. A conventional high-powered LED or laser light emitter (not shown) light emission source is transmittively coupled via a fiber optic cable (404) to the light pipe (402), which directs the emitted light downward along the back face of the retractor blade (210). Optical coupling of light in this manner avoids potential exposure of the patient to electrical currents through the device. When the light source is energized, the emitted light is directed into and illuminates the surgical field.

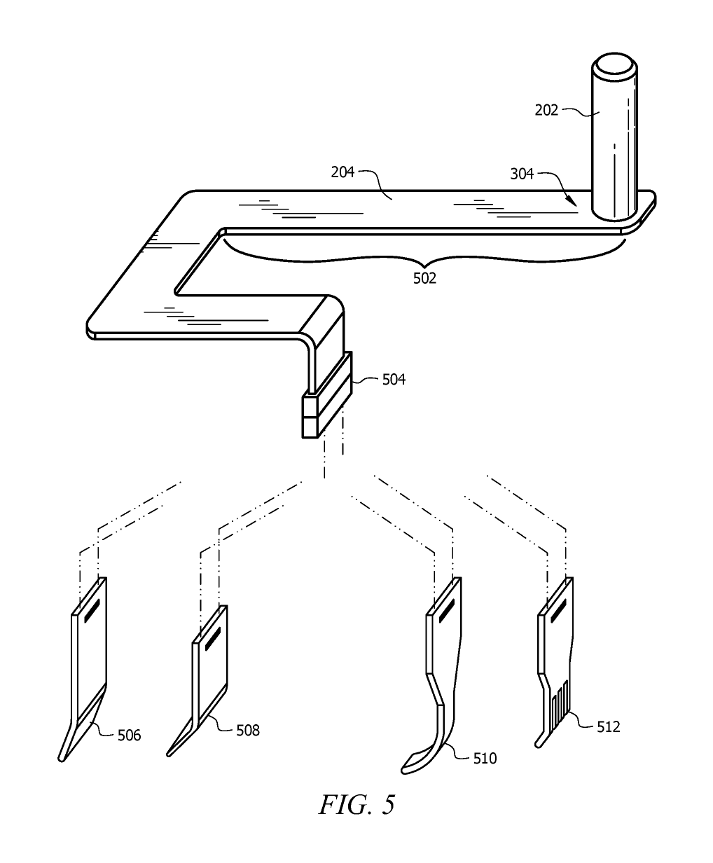

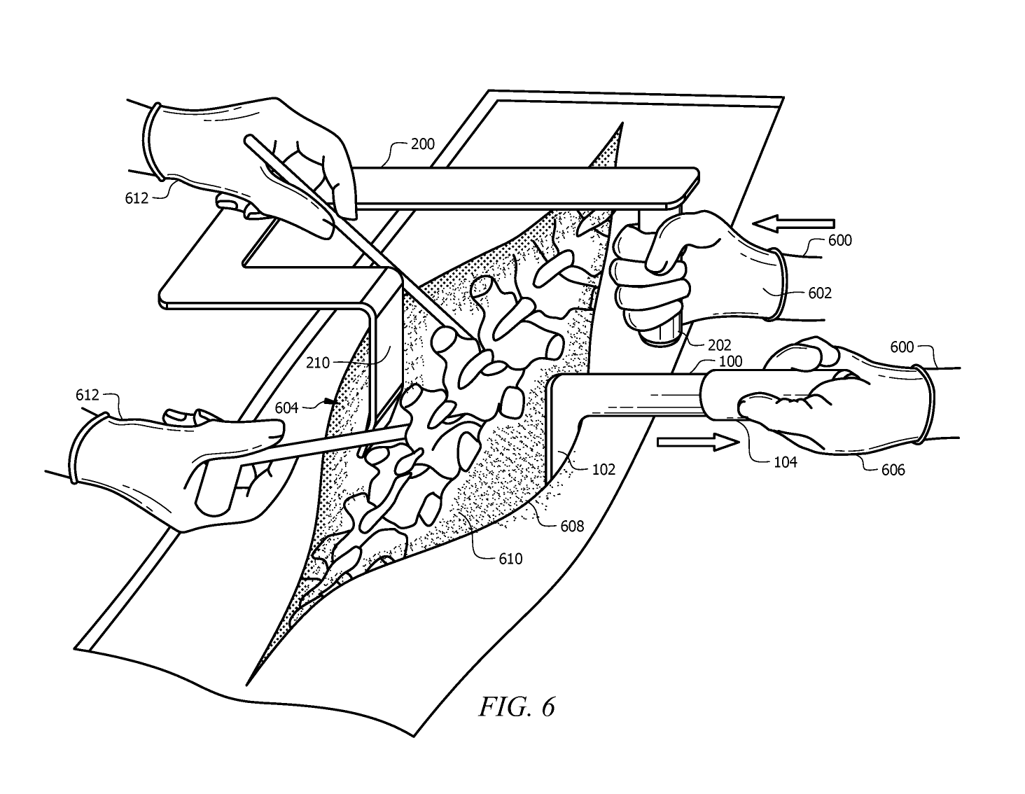

FIG. 5 is a depiction of another embodiment of the invention; highlighting an alternate handle and blade configuration. In this embodiment, the handle member (202) may be relocated to the top surface (304) of the compressive beam member (204) to afford greater clearance for the device to operate over a large patient. In yet another embodiment the first beam member, or compressive beam member, (204) is segmented and thereby adjustable in length (502) to allow the operator to adjust the device to ensure that the handle member (202) adequately spans the patient during use of the device during an operation to avoid interference with the patient.

Also, as depicted, is a releasable retention member (504) that allows selection of a retractor blade with desirable physical characteristics suitable for a given surgical procedure. For example, it might be necessary to utilize a long blade (506) for a deep incision; a shortened blade (508) for a shallower incision; a blade with a pin-point tip (506) for use in a small incision; a comb tip blade (512); or another custom blade shaped for a special purpose. Choice of blade characteristics is within the skill of one of ordinary skill based upon physical characteristics of the intended surgical field. The releasable retention member (504) releasably engages the desired blade, locking the blade in place for the surgical procedure.

FIG. 6 depicts use of the invention to establish an operative field using only a single surgical assistant. As depicted, the surgical assistant (600) grips the handle member (202) with the right hand (602) and places the retractor blade member (210) tissue engagement face (214) against the incised tissue edge on the contralateral (opposite) side (604) of the patient. The assistant also grips the handle (104) of a conventional retractor device (100) with the left hand (606) and places the conventional retractor blade (102) against the incised tissue edge on the near side (608) of the patient. By simultaneously pushing with the right hand (602) while pulling with the left hand (606), the incised tissue edges are spread apart as depicted, thereby establishing a surgical field (610) within which the surgeon (612) may operate. Thusly, a single surgical assistant (600) may establish an adequate surgical field (610) while remaining entirely on one side (the “lateral side”) of the patient opposite of that (the “contralateral side”) of the surgeon (612), affording the surgeon an essentially unencumbered surgical field (610) because of the displacement (308) between the compressive beam member and the tensile beam member of the contra retractor apparatus (200). The handle member (202) located on the lateral side of the patient is, therefore, operable on the blade member (210) on the contralateral side of the patient. Moreover, allowing the surgical assistant to displace the contralateral incised tissue edge (604) while remaining on the other side of the patient without reaching over the patient improves the surgical assistant's ability to maintain the surgical field (610) for an extended period without undue fatigue and physical discomfort.

The invention may be embodied in other specific forms without departing from the spirit or essential characteristics thereof. For example, while the embodiment of the invention described herein is arranged and depicted to be operable using the right hand, it is also envisioned that another embodiment is operable using the left hand. In this embodiment, the compressive and tensile beam member arrangement is swapped as in a mirror image, with the displacement between the compressive beam member and the tensile beam member affording an essentially unencumbered surgical field for a surgeon. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the recitation of method steps does not denote a limiting sequence for execution of the steps. Such method steps may therefore be performed in a sequence other than that recited unless the claim expressly states otherwise.

I claim:

1. A contra retractor apparatus for use in performing surgery on a patient, the apparatus comprising:
a handle member for gripping by an operator standing on a lateral side of a patient, the handle member connected by a plurality of beam members to a retractor blade member, the retractor blade member having a tissue engagement face for engaging an incised tissue on a contralateral side of the patient, the plurality of beam members configured such that the handle member is oriented on the lateral side of the patient and is operable on the blade member and on the incised tissue, which is oriented on the contralateral side of the patient, the contralateral incised tissue displaced in a contralateral direction when the handle member is pushed by the operator.

2. The apparatus of claim 1 wherein one or more of the plurality of beam members is adjustable in length.

3. The apparatus of claim 1 further comprising:
a shear beam member, wherein the length of the shear beam member is adapted to achieve the desired surgical field opening.

4. The apparatus of claim 1 further comprising:
a shear beam member connecting a compressive beam member to a tensile beam member, whereby a compressive force generated by the pushing motion is transferred as a shear force onto the shear beam member, and whereby the shear force is transferred as the tensile force onto the tensile beam member where the tensile force acts upon the blade member to impart force upon the contralateral incised tissue.

5. The apparatus of claim 1 further comprising:
a light pipe attached to the tensile beam member and adapted to accept a light emitted from a light source and to direct the emitted light downward along a back face of the retractor blade.

6. The apparatus of claim 1 further comprising:
a light pipe attached to the tensile beam member and adapted to accept a light emitted from a light source and to direct the emitted light downward along a back face of the retractor blade, the light pipe coupled to an optical fiber for transmitting emitted light from a light emission source.

7. The apparatus of claim 1 further comprising:
a releasable retention member for releasably retaining the retractor blade on the tensile beam member.

8. A contra retractor apparatus for use in performing surgery on a patient, the apparatus comprising:
at least one retractor blade member having and a back face and a tissue engagement face, the tissue engagement face for engaging an incised tissue of a patient; and
a handle member for gripping, the handle member connected to the at least one retractor blade member through a plurality of beam members, the plurality of beam members including a compressive beam member and a tensile beam member configured such that a user pushing on the handle member imparts a compressive force onto the compressive beam member, which is transferred as a tensile force onto the tensile beam member to act on the retractor blade member thereby imparting force on an engaged tissue of a patient.

9. The apparatus of claim 8 wherein one or more of the plurality of beam members is adjustable in length.

10. The apparatus of claim 8 further comprising:
a shear beam member connecting the compressive beam member to the tensile beam member, whereby the compressive force is transferred as a shear force onto the shear beam member, and whereby the shear force is transferred as the tensile force onto the tensile beam member.

11. The apparatus of claim 8 further comprising:
a shear beam member connecting the compressive beam member to the tensile beam member, wherein the length of the shear beam member is adapted to achieve the desired surgical field opening.

12. The apparatus of claim 8 further comprising:
a light pipe attached to the tensile beam member and adapted to accept light emitted from a light source and to direct the emitted light downward along the back face of the retractor blade.

13. The apparatus of claim 8 further comprising:
a light pipe attached to the tensile beam member and adapted to accept light emitted from a light source and to direct the emitted light downward along the back face of the retractor blade, the light pipe coupled to an optical fiber for transmitting emitted light from a light emission source.

14. The apparatus of claim 8 further comprising:
a releasable retention member for releasably retaining the retractor blade on the tensile beam member.

15. A contra retractor apparatus for use in performing surgery on a patient, the apparatus comprising:
a handle member for gripping, the handle member connected to a first beam member, the first beam member connected to a second beam member, and the second beam member connected to a third beam member, the third beam member attached to at least one retractor blade member, the retractor blade member having a back face an opposing tissue engagement face, the tissue engagement face for engaging the tissue of an incision on a patient,
wherein the first, second, and third beam members are configured such that when the retractor blade member tissue engagement face is engaged with an incised tissue on a patient, a compressive force imparted with the handle member onto the first beam member is transferred as a shear force onto the second beam member and is subsequently transferred as a tensile force onto the third beam member to act on the retractor blade member.

16. The apparatus of claim 15 wherein one or more of the beam members is adjustable in length.

17. The apparatus of claim 15, wherein the length of the second beam member is adapted to achieve the desired surgical field opening.

18. The apparatus of claim 15 further comprising:
a light pipe attached to the third beam member and adapted to accept light emitted from a light source and to direct the emitted light downward along the back face of the retractor blade.

19. The apparatus of claim 15 further comprising:
a light pipe attached to the third beam member and adapted to accept light emitted from a light source and to direct the emitted light downward along the back face of the retractor blade, the light pipe coupled to an optical fiber for transmitting emitted light from a light emission source.

20. The apparatus of claim 15 further comprising:
a releasable retention member for releasably retaining the retractor blade on the third beam member.
